(19) United States
(12) Patent Application Publication
(10) Pub. No.: US 2025/0233210 A1
(43) Pub. Date: Jul. 17, 2025

(54) NON-AQUEOUS ELECTROLYTES FOR ELECTROCHEMICAL CELLS

(71) Applicant: Sumitomo Chemical Co., Ltd, Tokyo (JP)

(72) Inventors: Florence BOURCET, Godmanchester (GB); Thomas Johannes KUGLER, Godmanchester (GB); Konrad ZALEWSKI, Godmanchester (GB)

(73) Assignee: Sumitomo Chemical Co., Ltd, Tokyo (JP)

(21) Appl. No.: 18/852,999

(22) PCT Filed: Mar. 30, 2023
(86) PCT No.: PCT/EP2023/058425

§ 371 Date: Sep. 30, 2024

(30) Foreign Application Priority Data
Mar. 31, 2022 (GB) 2204653.6

(51) Int. Cl. H01M 10/0568; H01M 10/0569

(52) U.S. Cl. CPC H01M 10/0568; H01M 10/0569; H01M 2300/0028; H01M 2300/0051

(57) ABSTRACT

A compound of formula (I): Core is a core group; X is Al or B; R¹ in each occurrence is independently a substituent and two R¹ groups may be linked to form a ring; L is a linking group; M⁺ is a cation; and n is at least 2. The compound may be used in a battery, e.g. a metal battery.

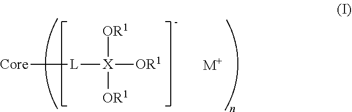

### BACKGROUND

US 2019/0185492 discloses ionic covalent frameworks comprising tetra-coordinated borate linkages.

Aubrey et al, “Dependence of Linker Length and Composition on Ionic Conductivity and Lithium Deposition in Single-Ion Conducting Network Polymers”, Macromolecules 2021 54(16), 7582-7589 discloses a polymer containing borate groups linked by oligoethylene glycoxide linkers.

JPH08301879 discloses compounds of formula (II) and (III):

Li⁺(R¹X)₃Al⁻XR²XAl⁻(R¹X)₃Li⁺ (II)

Li⁺(R¹X)₃Al⁻XR³(XAl⁻(R¹X)₃Li⁺)₂ (III)

### SUMMARY

In some embodiments, the present disclosure provides a compound of formula (I):

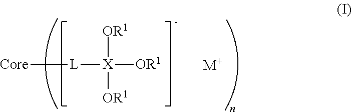

wherein:
Core is a core group;
X is Al or B;
R¹ in each occurrence is independently a substituent and two R¹ groups may be linked to form a ring;
L is a linking group;
M⁺ is a cation; and n is at least 2.

In some embodiments, none of the R¹ groups are linked. In these embodiments, optionally each R¹ is independently a C₁₋₂₀ alkyl group wherein one or more non-adjacent C atoms of the alkyl group may be replaced with O, S, CO or COO and one or more H atoms of the alkyl group may be replaced with F.

Optionally, each R¹ is independently selected from alkyl and alkyl ether groups wherein one or more H atoms may be replaced with F.

Optionally, each R¹ is the same.

Optionally, the compound contains at least 2 different R¹ groups.

In some embodiments, two R¹ groups of formula (I) are linked and the compound of formula (I) has formula (Ia):

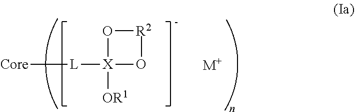

wherein R² in each occurrence is independently a divalent organic group.

### Optionally, L is O.

Optionally, n is 2, 3 or 4.

Optionally, Core is selected from the group consisting of:
(Ar²)p wherein p is at least 1, preferably 1, 2 or 3, and Ar² in each occurrence is indpendently an arylene or heteroarylene group which is unsubstituted or substituted with one or more substituents;
—N(R⁸)x(R⁹)y wherein R⁸ is a divalent organic group; R⁹ is a monovalent group; x is 2 or 3; and y is 3-x; and
Ak wherein Ak is a linear or branched C₁₋₂₀ alkylene wherein one or more non-adjacent C atoms may be replaced with O, S, CO or COO and one or more C atoms may be replaced with arylene or heteroarylene.

Optionally, M⁺ is an alkali metal ion, preferably a lithium ion.

Optionally, M⁺ is a solvated cation.

Optionally, the solvate of the solvent is selected from solvents comprising at least one ether group.

Optionally, the solvate: M⁺ molar ratio is no more than 10:1.

Optionally, the solvate: M⁺ molar ratio is at least 0.5:1.

In some embodiments, the present disclosure provides a formulation comprising a compound of formula (I) and a solvent. Optionally, the solvate: M⁺ molar ratio of the formulation is no more than 20:1.

In some embodiments, the present disclosure provides a method of forming a compound as described herein comprising reacting a compound of formula (II) and both compounds of formula (III) and (IV):

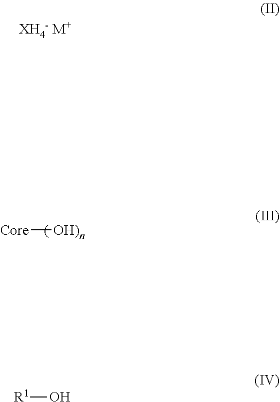

Optionally, the reaction is carried out in a reaction mixture comprising the solvent of the solvate as described above.

Optionally, the method comprises at least partially replacing the solvent of a solvate of the compound with another solvent.

In some embodiments, the present disclosure provides a metal battery or metal ion battery comprising an anode, a cathode and a compound as described herein disposed between the anode and the cathode.

Optionally, a solvate: M⁺ molar ratio of the battery is no more than 10:1.

Optionally, the metal battery comprises an anode protection layer comprising the compound or polymer disposed between the anode and cathode.

### DESCRIPTION OF DRAWINGS

FIG. 1 is a schematic illustration of a battery according to some embodiments of the present disclosure having a separator comprising a compound as described herein;

FIG. 2 is a schematic illustration of a battery according to some embodiments of the present disclosure having an anode protection layer comprising a compound as described herein;

FIG. 3 is a NMR spectrum of a compound according to an embodiment of the present disclosure;

FIG. 4 shows Nyquist plots for a cell according to an embodiment of the present disclosure and a comparative cell;

FIG. 5 shows current profiles applied to tested cells;

FIGS. 6A and 6B show, respectively, EIS measurement (left) and DC measurement for a cell according to an embodiment of the present disclosure; and

FIGS. 7A and 7B show, respectively, EIS measurement (left) and DC measurement for a comparative cell.

The drawings are not drawn to scale and have various viewpoints and perspectives. The drawings are some implementations and examples. While the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers may be present. References to a layer “on” another layer when used in this application means that the layers are in direct contact. References to an element of the Periodic Table include any isotopes of that element.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

In some embodiments, the present disclosure provides compounds of formula (I):

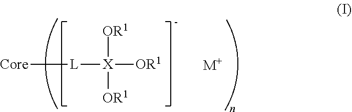

X is Al or B.
R¹ in each occurrence is independently a substituent and two R¹ groups may be linked to form a ring.
M⁺ is a cation.
L is a linking group, optionally O, S or NR⁴ wherein R⁴ is H or a substituent.

Core is a core unit to which n aluminate or borate groups are attached.
n is at least 2, optionally 2-6, preferably 2, 3 or 4.

Preferably. L is O.

In the case where L is NR⁴, R⁴ is preferably selected from H; optionally substituted aryl; optionally substituted heteroaryl; and C₁₋₁₂ alkyl wherein one or more non-adjacent, non-terminal C atoms of the C₁₋₁₂ alkyl may be replaced with O, S, CO, COO or NR⁵ and one or more H atoms of the C₁₋₁₂ alkyl may be replaced with F, wherein R⁵ is H or a substituent, preferably H or a C₁₋₆ alkyl.

An aryl or heteroaryl group R⁴ is preferably phenyl. Where present, substituents of an aryl or heteroaryl group R⁴ are preferably selected from F, CN, NO₂ and C₁₋₁₂ alkyl wherein one or more non-adjacent, non-terminal C atoms of the C₁₋₁₂ alkyl may be replaced with O, S, NR⁵, CO or COO and one or more H atoms may be replaced with F.

Preferably, Core groups are selected from:
(Ar²)p wherein p is at least 1, preferably 1, 2 or 3, and Ar² in each occurrence is independently an arylene or heteroarylene group which is unsubstituted or substituted with one or more substituents;
—N(R⁸)x(R⁹)y wherein R⁸ is a divalent organic group; R⁹ is a monovalent group; x is 2 or 3; and y is 3-x; and
Ak wherein Ak is a linear or branched C₁₋₂₀ alkylene wherein one or more non-adjacent C atoms may be replaced with O, S, CO or COO and one or more C atoms may be replaced with arylene or heteroarylene, preferably phenylene.

Ar² in each occurrence is preferably selected from a C₆₋₁₀ arylene or a 5- or 6-membered heteroarylene ring.

Exemplary Core groups of formula (Ar²)p include benzene, biphenylene and pyridine, each of which may be unsubstituted or substituted with one or more non-ionic substituents.

Exemplary non-ionic substituents of Ar² are F, CN, NO₂, N(R¹⁰)₂ wherein R¹⁰ in each occurrence is independently H or aryl, preferably phenyl, and C₁₋₁₂ alkyl wherein one or more non-adjacent, non-terminal C atoms may be replaced with O, S, NR⁷, CO or COO and one or more H atoms may be replaced with F.

R⁷ is H or a substituent and is preferably selected from groups as defined for R⁴.

For Core groups of formula N(R⁸)x(R⁹)y, each R⁸, which may be the same or different, preferably the same, is preferably selected from optionally substituted arylene, preferably benzene; optionally substituted heteroarylene; and C₁₋₁₂ alkylene wherein one or more non-adjacent C atoms may be replaced with O, S, CO, COO or arylene and one or more H atoms may be replaced with F.

In the case where y is 1, R⁹ is preferably selected from optionally substituted aryl, preferably benzene; optionally substituted heteroaryl; and C₁₋₁₂ alkylene wherein one or more non-adjacent C atoms may be replaced with O, S, CO, COO, NR⁵ or arylene and one or more H atoms may be replaced with F.

Exemplary substituents of an aryl(ene) or heteroaryl(ene) group of R⁸ or R⁹ are F, CN, NO₂ and C₁₋₁₂ alkyl wherein one or more non-adjacent C atoms may be replaced with O, S, CO, COO or NR⁵.

Exemplary Core groups of formula N(R⁸)x(R⁹)y include tri (C₁₋₁₂)alkylamine wherein at least two of the three alkyl groups are bound to a group L of formula (I).

Exemplary Core groups Ak include 2-methyl propane bound to 2 or 3 groups L; tert-butane bound to 2 or 3 groups L; neopentane bound to 2, 3, or 4 groups L; and a group of formula C(CH₂CH₂OCH₂CH₂—)₄ bound to 4 groups L. Halogenated, more preferably fluorinated Core groups Ak are preferred.

By “terminal C atom” of an alkyl group as used herein is meant the C atom of the methyl group or methyl groups at the chain end or chain ends of a linear or branched alkyl, respectively.

Core is non-polymeric. By “non-polymeric” as used herein is meant that Core is not polydisperse. Preferably, Core has a molecular weight of no more than 800 Daltons.

In some preferred embodiments, none of the R¹ groups are linked. Optionally according to these embodiments, each R¹ is independently a C₁₋₂₀ alkyl group wherein one or more non-adjacent C atoms of the alkyl group may be replaced with O, S, CO or COO and one or more H atoms of the alkyl group may be replaced with F.

Preferred R¹ groups include C₁₋₂₀ alkyl wherein one or more C atoms other than the C atom bound to O of OR¹ or a terminal C atom may be replaced with O, and one or more H atoms may be replaced by F.

In some embodiments, each R¹ is the same.

In some embodiments, the compound contains two or more different R¹ groups.

In some embodiments, two R¹ groups of formula (I) are linked and the compound of formula (I) has formula (Ia):

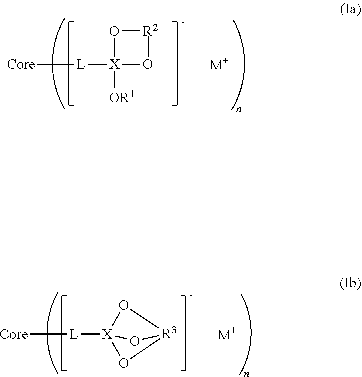

wherein R² in each occurrence is independently a divalent group and R³ is a trivalent organic group.

Optionally, R² is selected from a C₆₋₂₀ arylene group, e.g. 1,2-phenylene, which may be unsubstituted or substituted with one or more substituents; a bi-arylene group, for example 2,2′-linked biphenylene; ethylene; propylene; and diethyleneamine, each of which may be unsubstituted or substituted with one or more substituents. Optionally, substituents are selected from F alkyl wherein one or more non-terminal C atoms of the C₁₋₁₂ alkyl may be replaced with F and one or more C atoms of the C₁₋₁₂ alkyl may be replaced with O.

Optionally, R³ is a trivalent amine, for example triethylene amine; a trivalent group-(Ar²)p or a trivalent group Ak.

Preferably, M⁺ is an alkali metal cation, more preferably a lithium cation.

Preferably, M⁺ is a solvated cation.

Preferably, the solvent of the solvate is selected from solvents comprising at least one ether group.

Preferably, the solvent contains two or more groups capable of coordinating to the metal cation.

The solvent may be selected from: linear and cyclic compounds containing one or more ether groups and, optionally, one or more groups selected from hydroxyl and carboxylate groups; and solvents containing carbonate groups, for example C₂₋₁₀ alkylene carbonates and di(C₁₋₁₀ alkyl) carbonates.

Exemplary solvents include, without limitation, propylene carbonate, ethylene carbonate, dimethyl carbonate, tetrahydrofuran, dimethoxyethane (DME), diglyme (diethylene glycol dimethyl ether), triglyme (triethylene glycol dimethyl ether), tetraglyme (tetraethylene glycol dimethyl ether) and crown ethers, for example 12-Crown-4 and 1-aza-12-Crown-4.

The compound may contain more than one solvent of a solvate.

Optionally, a battery containing a compound of formula (I) contains no solvent, or only a small amount of solvent, preferably no more than 20 moles of solvate per mole of M⁺. The presence of a small amount of solvent has been found to significantly increase the ionic conductivity of the compound of formula (I). This increase is attributed to solvation of the cation; where solvation takes place, it will be understood that M⁺ is solvated by at least some but not necessarily all of the solvent present. The presence of a small amount of organic solvent such as an ether- or carbonate-containing solvent may enhance ionic conductivity whilst significantly reducing flammability as compared to an ionic compound dissolved in a large volume of such a solvent. In some embodiments, all solvent is solvating solvent only. In some embodiments, solvating solvent and additional free (non-solvating) solvent is present.

Accordingly, a formulation comprising a solvent and a compound of formula (I) preferably comprises no more than 20 moles of solvent, optionally no more than 10 moles or no more than 8 or 6 moles of solvent, per mole of M⁺. Preferably, the formulation contains at least 0.5 moles or at least 1 mol of solvent per mole of M⁺.

The amount of solvating solvent in a compound of formula (I) may be determined from a ¹H NMR spectrum of the compound following vacuum treatment to remove free (non-solvating) solvent by integration of 1H NMR peaks corresponding to the solvent and peaks corresponding to the groups —O—R¹.

The compound of formula (I) may be formed by reacting a compound of formula (II) and both compounds of formula (III) and (IV):

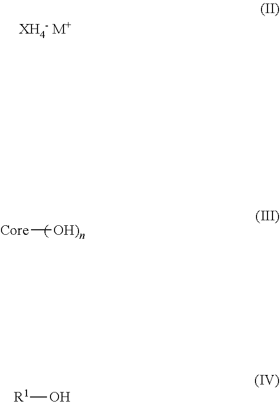

Exemplary compounds of formula (I) include, without limitation, lithium aluminium hydride (LiAlH₄), lithium borohydride (LiBH₄).

If the metal cation M⁺ is a solvated cation then in some embodiments the solvent of the solvate is present in the reaction mixture containing the compound of formula (II) and the compound of formula (III).

In some embodiments, the solvent of a compound of formula (I) containing a solvated cation may be replaced with a different solvent. Methods of changing the solvent of a solvate include, without limitation, driving off a solvent of a compound of formula (I) by heat treatment and replacing it with another solvent capable of solvating the cation; and contacting a compound of formula (I) with a solvent which coordinates more strongly to the cation than an existing solvating solvent, for example by treating a compound of formula (I) having a monodentate solvate solvent with a bi-dentate or higher-dentate ligand.

### Applications

A single-ion conducting compound of formula (I) as described herein may be provided in a rechargeable battery cell. The battery may be, without limitation, a metal battery or a metal ion battery, for example a lithium battery or a lithium ion battery.

The compound of formula (I) may be a component of a composite comprising one or more additional materials, for example one or more polymers. A composition comprising a compound of formula (I) and a polymer may form a gel.

A layer comprising or consisting of the compound of formula (I) may be formed by depositing a formulation containing the material dissolved or dispersed in a solvent or solvent mixture.

Optionally, a battery comprising the compound of formula (I) contains no more than 20 moles of solvent per mole of M⁺, optionally no more than 10 moles of solvent per mole of M⁺, and/or no solvent other than any solvating solvent as described herein.

The formulation may comprise a polymer additional material dissolved in the solvent or solvents.

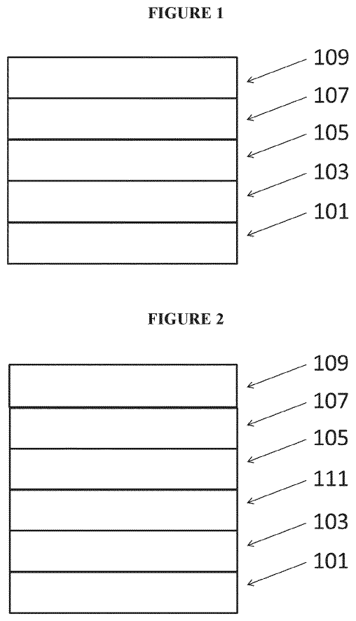

FIG. 1 illustrates a battery comprising an anode current collector 101 carrying an anode 103 on a surface thereof; a cathode current collector 109 having a cathode 107 disposed on a surface thereof; and a separator 105 disposed between the anode and cathode. The separator comprises or consists of a compound of formula (I). Preferably, the separator comprises no more than 10 moles of solvent per mole of M⁺ and/or no solvent other than any solvating solvent as described herein.

The battery may be a metal battery. The battery may be a metal ion battery.

In the case of a metal battery, the anode is a layer of metal (e.g. lithium) which is formed over the anode current collector during charging of the battery and which is stripped during discharge of the battery.

In the case of a metal ion battery, the anode comprises an active material, e.g. graphite, for absorption of the metal ions.

The cathode may be selected from any cathode known to the skilled person.

The anode and cathode current collectors may be any suitable conductive material known to the skilled person, e.g. one or more layers of metal or metal alloy such as aluminium or copper.

FIG. 1 illustrates a battery in which the anode and cathode are separated only by a separator. In other embodiments, one or more further layers may be disposed between the anode and the separator and/or the cathode and the separator.

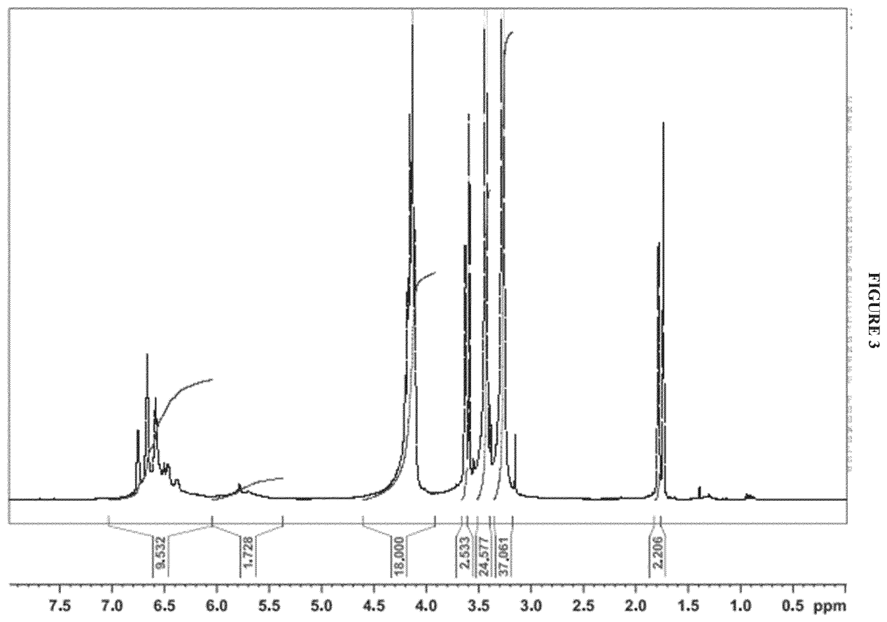

FIG. 2 illustrates a battery, preferably a metal battery, comprising an anode current collector 101 carrying an anode 103 on a surface thereof; a cathode current collector 109 having a cathode 107 disposed on a surface thereof; a separator 105 disposed between the anode and cathode; and an anode protection layer 111 disposed between anode and the separator. The separator may comprise or consist of a compound as described herein or may be any other separator known to the skilled person, for example a porous polymer having a liquid electrolyte absorbed therein. The anode protection layer comprises or consist of a compound of formula (I) as described herein. The anode protection layer may prevent or retard formation of lithium metal dendrites of a metal battery.

### EXAMPLES

### Compound Example 1

Compound Example 1 was prepared according to the following reaction scheme:

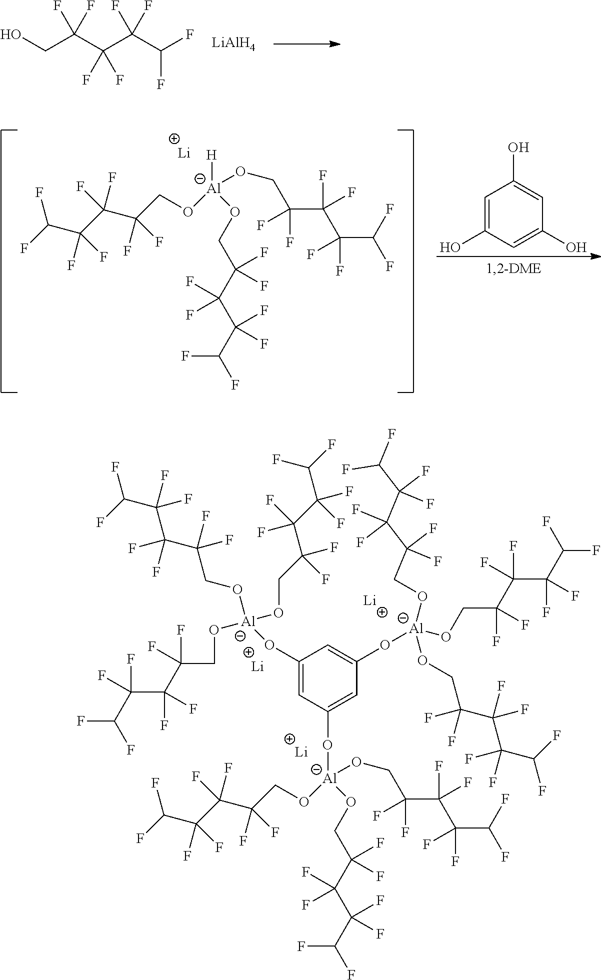

To a solution of lithium aluminium hydride (9 ml, 9.0 mmol, 1.0 M in tetrahydrofuran) was added drop wise a solution of 2,2,3,3,4,4,5,5-octafluoro-1-pentanol (3.74 ml, 27.0 ml) in anhydrous 1,2-dimethoxyethane (5 ml) at room temperature. The resulting solution was stirred for 30 minutes. 1,3,5-trihydroxybenzene (0.377 g, 3.0 mmol) was dissolved in 1,2-dimethoxyethane (5 ml) and added to the stirred reaction mixture at room temperature. The reaction mixture was stirred at room temperature for 1 hour then it was heated up to 60° C. for 2 hours. The mixture was cooled down to room temperature and left over-night under nitrogen. Extra lithium aluminium hydride (0.4 ml, 0.4 mmol, 1.0 M in tetrahydrofuran) solution was added to the reaction mixture at room temperature and mixture was heated up to 60° C. for 2 hours then cooled down to room temperature.

Excess solvent was removed under reduced pressure (3.0×10−2 mbar) at room temperature to yield a thick opaque oil.

With reference to FIG. 3, the NMR spectrum of the product suggests that there is a mixture of compounds present in the product. From integration of NMR peaks, it was calculated that for three molecules of 2,2,3,3,4,4,5,5-octafluoro-1-pentanol in the product mixture there is 2.0 molecules of 1,2-dimethoxyethane and 0.75 molecules of tetrahydrofuran present as residual solvent, which are believed to solvate the lithium ions of Compound Example 1.

### Cell Example 1

EIS measurements were conducted on 2032-type coin cell devices (casings purchased from Cambridge Energy Solutions) in which the following layers were placed in sequence between the coin cell bottom and top:
a stainless steel disk
a lithium disk
a fluorosilicone stencil (purchased from Silex Silicones) shaped as a disk of 15.5 mm diameter, with a circular hole of 5 mm diameter cut in its middle,
30 microlitres of electrolyte of Compound Example 1 filled in the circular hole,
a lithium disk
a stainless steel disk
a wave spring.

The coin cell was crimped to give a cell in which the thickness of the stencil was 360 microns.

The electrolyte consisted of Compound Example 1, DME (2.05 moles per mole of Li⁺) and tetrahydrofuran (0.18 moles per more of Li⁺).

### Comparative Cell 1

A cell was prepared as described for Cell Example 1 except that compound LiAl(OFP)₄ was used in place of Compound Example 1.

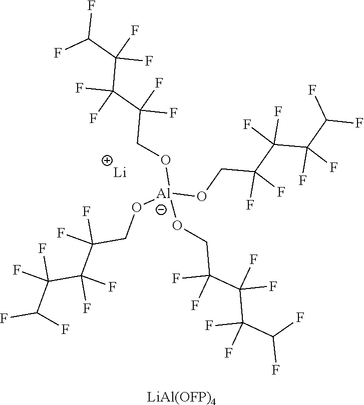

The LiAl(OFP)₄ contained 0.85 molecules of DME and 1.13 molecules of propylene carbonate per lithium cation.

### Electrochemical Impedance Spectroscopy (EIS)

EIS measurements on the coin cells were conducted at room temperature. The EIS measurements were take using a potentiostat (Interface 1010E, Gamry Instruments) over a frequency range of 1 Hz to 1 MHz with an amplitude of 5 mV.

Ionic conductivity was calculated using the following formula:

$$σ = \frac{l}{A \cdot R}$$

where l is the thickness of the material between the two stainless disks which corresponds to the 360 micron of the crimped fluorinated separator, A is the area of the hole were the material was deposited and R is the impedance.

The impedance of the cell was determined by determining the intercept of the 1^{st} semi-circle on the x-axis of the Nyquist plot.

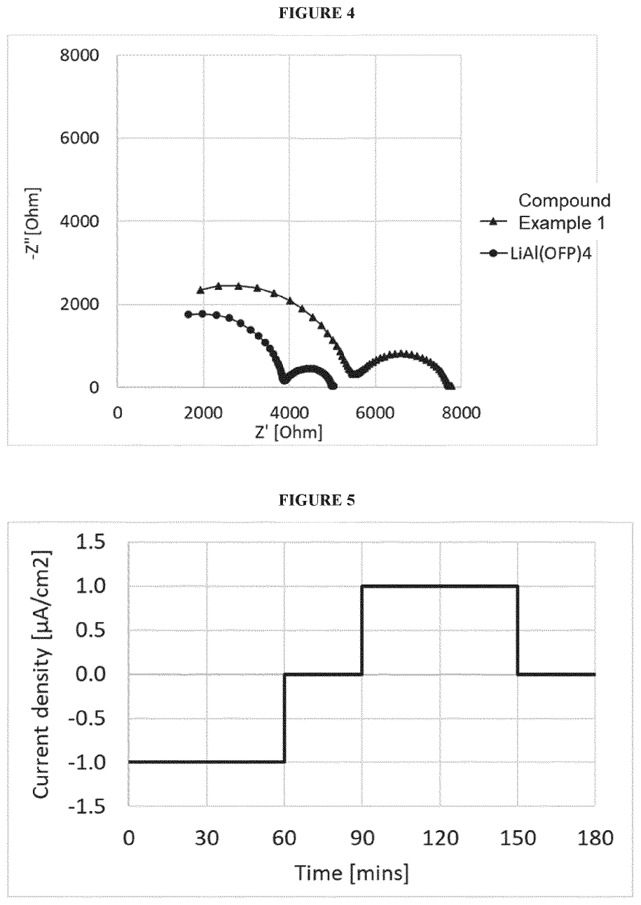

The Nyquist plots for Cell Example 1 and Comparative Cell 1 are shown in FIG. 4. As shown in Table 1, ionic conductivities for the two compounds are of the same order of magnitude.

TABLE 1

| Material | Ionic conductivity (S/cm) |
|---|---|
| Compound Example 1 | 3.3 × 10⁻⁵ |
| LiAl(OFP)₄ Comparative | 4.7 × 10⁻⁵ |

### Lithium Transference Number (LTN)

The LTN measurements were conducted on the cells described herein, using Evans's method as set out in J. Evans et al., Polymer, 1987, Vol 28.

Prior to the LTN measurements, devices were pre-conditioned by applying Lithium plating and stripping cycles on the battery testing system (Arbin Instruments) for 18 hours at 1 μA/cm².

Constant currents (negative or positive) were applied for 60 mins, with 30 mins rest periods in-between the plating and stripping intervals, according to the profile shown in FIG. 5. Following pre-conditioning, measurements for determining LTN were taken as follows:
1. A 1st EIS spectrum was measured.
2. This was followed by a DC current measurement (at a constant voltage of 10 mV applied for 120 mins) on the battery testing system (Arbin Instruments).
3. This was followed by a 2nd EIS measurement.

EIS measurements were performed as described above.

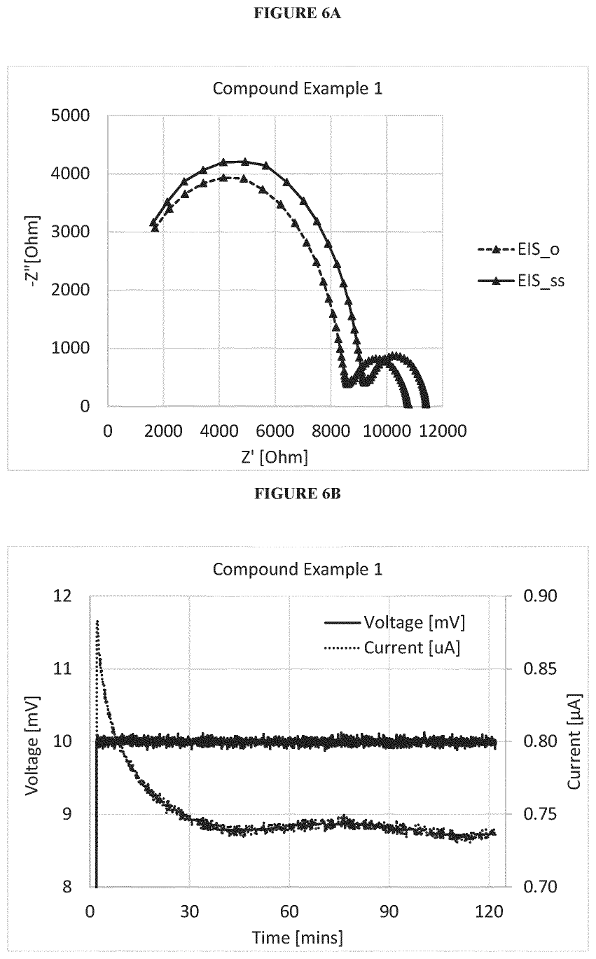

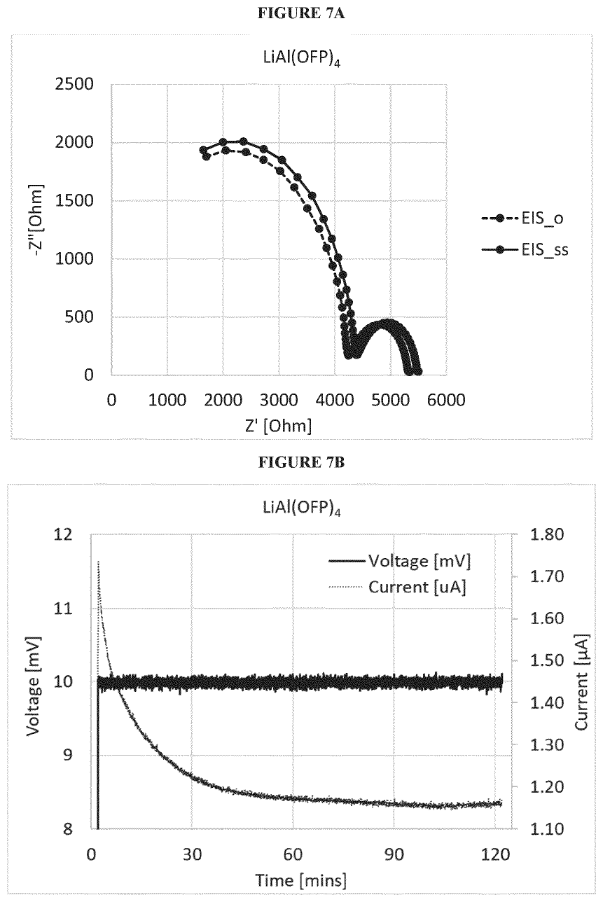

The LTN was calculated according to the following formula, based on the model developed by Evans et al (ibid):

$$LTN = \frac{{I}^{s s} ( ΔV - {I}^{0} {R}^{0} )}{{I}^{0} ( ΔV - {I}^{s} {R}^{s} )}$$

Where, with reference to FIGS. 6-7:
R⁰ is the initial charge transfer impedance, which is the width of the second half circle in the 1st EIS spectrum,

R^{ss} is the steady state charge transfer impedance which is the width of the second half circle in the 2nd EIS spectrum (after a 10 mV DC bias was applied),
I⁰ is the initial current taken when the voltage is stepped up from the open circuit voltage to 10 mV, and
I^{ss} is the steady state current taken at 120 mins (at the end of the DC measurement).

Parameters extracted from these plots are summarised in Table 2, together with the calculated LTN values for Compound Example 1 and LiAl(OFP)₄, showing higher LTN for Compound Example 1.

TABLE 2

| | Compound | |
|---|---|---|
| Parameter | Example 1 | LiAl(OFP)₄ |
| ΔV [V] | 1.00E−02 | 1.00E−02 |
| R^{o} [Ohm] | 2237 | 1150 |
| R^{ss} [Ohm] | 2288 | 1155 |
| I^{o} [A] | 8.83E−07 | 1.74e−06 |
| I^{ss} [A] | 7.36E−07 | 1.16e−06 |
| LTN | 0.80 | 0.62 |

1. A compound of formula (I): 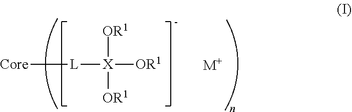
wherein:
Core is a core group;
X is Al or B;
R¹ in each occurrence is independently a substituent and two R¹ groups may be linked to form a ring;
L is a linking group;
M⁺ is a cation; and
n is at least 2.

2. The compound according to claim 1 wherein none of the R¹ groups are linked.

3. The compound according to claim 2 wherein each R¹ is independently a C₁₋₂₀ alkyl group wherein one or more non-adjacent C atoms of the alkyl group may be replaced with O, S, CO or COO and one or more H atoms of the alkyl group may be replaced with F.

4. The compound according to claim 3 wherein each R¹ is independently selected from alkyl and alkyl ether groups wherein one or more H atoms may be replaced with F.

5. The compound according to claim 2 wherein each R¹ is the same.

6. The compound according to claim 1 wherein the compound contains at least 2 different R¹ groups.

7. The compound according to claim 1 wherein two R¹ groups of formula (I) are linked and the compound of formula (I) has formula (Ia): 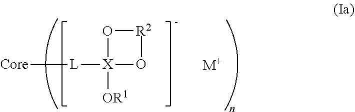
wherein R² in each occurrence is independently a divalent organic group.

8. The compound according to claim 1 wherein L is O.

9. The compound according to claim 1 wherein n is 2, 3 or 4.

10. The compound according to claim 1 wherein Core is selected from the group consisting of:
(Ar²)p wherein p is at least 1, preferably 1, 2 or 3, and Ar² in each occurrence is independently an arylene or heteroarylene group which is unsubstituted or substituted with one or more substituents;
N(R⁸)x(R⁹) y wherein R⁸ is a divalent organic group; R⁹ is a monovalent group; x is 2 or 3; and y is 3-x; and
Ak wherein Ak is a linear or branched C₁₋₂₀ alkylene wherein one or more non-adjacent C atoms may be replaced with O, S, CO or COO and one or more C atoms may be replaced with arylene or heteroarylene.

11. The compound according to claim 1 wherein M⁺ is an alkali metal ion.

12. The compound according to claim 11 wherein M⁺ is a lithium ion.

13. The compound according to claim 1 wherein M⁺ is a solvated cation.

14. The compound according to claim 13 wherein the solvate of the solvent is selected from solvents comprising at least one ether group and at least one carbonate group.

15. The compound according to claim 13 wherein the solvate: M⁺ molar ratio is no more than 10:1.

16. The compound according to claim 13 wherein the solvate: M⁺ molar ratio is at least 0.5:1.

17. A formulation comprising a compound according to claim 1 and a solvent.

18. The formulation according to claim 17 wherein the solvate: M⁺ molar ratio is no more than 20:1.

19. A method of forming a compound according to claim 1 comprising reacting a compound of formula (II) and both compound of formula (III) and 4:

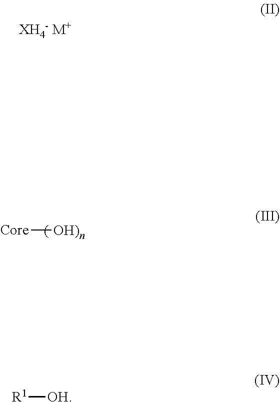

20. A method of forming a compound according to claim 1 comprising reacting a compound of formula (II) and both compound of formula (III) and 4: 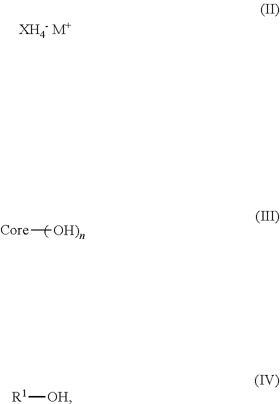
wherein the compound according to claim 1 is a solvated compound in which M+ is solvated by a solvent and the reaction is carried out in a reaction mixture comprising the solvent of the solvate.

21. A method comprising at least partially replacing the solvent of the solvate of the compound according to claim 15 with another solvent.

22. A metal battery or metal ion battery comprising an anode, a cathode and a compound according to claim 1 disposed between the anode and the cathode.

23. A metal battery or metal ion battery according to claim 22 wherein the battery comprises a solvent and wherein a solvent: M⁺ molar ratio of the battery is no more than 20:1.

24. A metal battery according to claim 22 comprising an anode protection layer comprising the compound of formula (I): 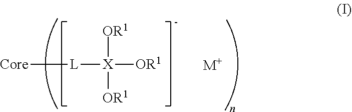
wherein:
Core is a core group:
X is Al or B;
R¹ in each occurrence is independently a substituent and two R¹ groups may be linked to form a ring;
L is a linking group;
M⁺ is a cation; and
n is at least 2 disposed between the anode and cathode.
